(19) United States
(12) Patent Application Publication
(10) Pub. No.: US 2014/0099296 A1
(43) Pub. Date: Apr. 10, 2014

(54) TOPICALLY OR ORALLY APPLIED ALOE, DISTILLED WATER, SODIUM CHLORIDE, AND LYSOZYME MIXTURE AND METHOD OF PRODUCING THE MIXTURE

(71) Applicant: Jeffrey Allen Covell, Port Saint Lucie, FL (US)

(72) Inventor: Jeffrey Allen Covell, Port Saint Lucie, FL (US)

(21) Appl. No.: 13/644,502

(22) Filed: Oct. 4, 2012

(51) Int. Cl. A61K 38/47; A61P 31/04; A61P 29/00; A61P 17/02

(52) U.S. Cl. 424/94.61

(57) ABSTRACT

A skin and/or oral treatment mixture for treating tattooed or pierced skin such as that resulting from a body piercing, oral piercing, or tattoo procedure includes non-iodized sodium chloride, distilled water, aloe, and lysozyme. The distilled water is preferably a product of vapor or steam distillation. The Aloe is preferably pure aloe barbadensis leaf extract. A method of producing a skin and/or oral treatment mixture includes the steps of providing a quantity of distilled water; adding a quantity of non-iodized sodium chloride; Heating the quantities of vapor distilled water and non-iodized sodium chloride to 70 degrees Celsius; Cooling the water/sodium mixture to 40 degrees celcius; adding a quantity of aloe barbadensis and a quantity of lysozyme; stirring the mixture. A method for treating the tattooed or pierced area of a persons skin includes applying to the pierced or tattooed area a distilled water, sodium chloride, aloe and lysozyme.

### BACKROUND—PRIOR ART

I, Jeffrey Covell, The inventor of this mixture have been in the body piercing and tattoo industry for over twenty years and the Owner of two professional tattoo and piercing establishments. In that time I have noticed several different industry wide prescribed ways for aftercare treatment of new body piercings and tattoos. All of these methods seemed to work haphazardly if at all. Hydrogen peroxide, methylate and even the current manufacturers of Saline rinses all left something to be desired as far as quicker and safer heal durations without risk of infection.

### ADVANTAGES

This application produces a mixture that is far superior to any prior art, and made from all natural ingredients which provide natural antimicrobials, antiseptics, and antibacterial protection and also suitable for cleansing dried discharge and lymph secretions in a safe manner and meanwhile reducing the chance of infection, epidermal scarification, bumps, cellulitis, itching, swelling, and increased circulatory flow. This method of topical or oral application and healing with this mixture is suitable for the healing of tattoos, oral, and body piercings, infected gums, mouth sores, sore throat, tonsillitis, abscessed teeth, cuts, scrapes, abrasions and any superficial wounds to the epidermal layer of skin. Specific prior art, which this application cures, is an application by Edward Kolos. Mr. Kolos, neither a body piercer nor tattoo artist, attempts to provide a specific mixture of reconstituted ocean water using the ingredients of sea salt, the product of reverse osmosis of water, and lysozyme. The said prior art does nothing exceptional to heal a tattoo or body piercing any faster than applying a simple saline solution. The current application solves this issue by the presence of Aloe Barbadensis in its purest form. Aloe is well known for it's anti-inflammatory, and anti-microbial properties. The use of Aloe in conjunction with the lysozyme creates a reaction whereby the aloe actually helps carry the lysozyme to the cell walls of any bacteria, which may be present.

### THE SUMMARY

The present invention is directed to a topically or orally applied aloe barbadensis leaf, non-iodized sodium chloride, lysozyme, and distilled water mixture whose effectiveness and welfare are based on its innovative composition as well as the method of treating a pierced or tattooed area of the skin.

A skin treatment mixture is provided, including a quantity of distilled water, a quantity of aloe barbadensis leaf extract, non-iodized sodium chloride, the natural enzyme lysozyme.

A method of producing the skin treatment mixture is given, including the steps of adding a quantity of non-iodized sodium chloride to vapor distilled water and mixing the ingredients after heating the ingredients to 70 degrees Celsius. Mixture is maintained at the said temperature for 20 minutes to ensure proper bacterial elimination. The mixture is then cooled to 40 degrees Celsius and then pure aloe extract and lysozyme are added. Mixture is then mixed and cooled. Finished mixture is then topically or orally applied depending on specific use.

### DRAWINGS—FIGURES

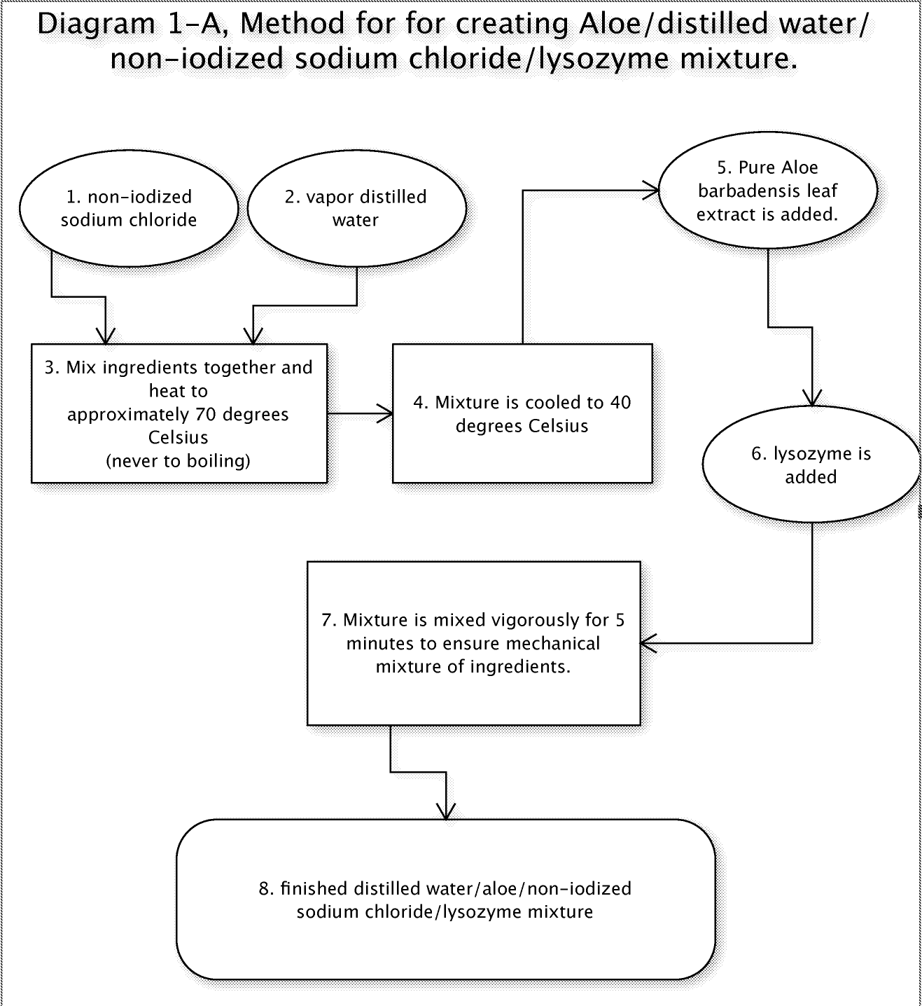

Please refer to FIG. 1-A which is a flow chart of the novel process of making the present topically or orally applied aloe barbadensis leaf, non-iodized sodium chloride, lysozyme, and distilled water mixture.

### A. REFERENCE NUMERALS

1. Non-iodized sodium chloride

2. Vapor distilled water

3. Mix ingredients together and heat to approximately 70 degrees Celsius (never to boiling)

4. Mixture is cooled to 40 degrees Celsius

5. Pure Aloe barbadensis leaf extract is added.

6. Lysozyme is added

7. Mixture is mixed vigorously for 5 minutes to ensure mechanical mixture of ingredients.

8. Finished distilled water/aloe/non-iodized sodium chloride/lysozyme mixture.

### DETAILED DESCRIPTION

As required, detailed embodiments of the present invention are disclosed herein; however, it is to be understood that the disclosed embodiments are merely exemplary of the invention that may be embodied in many forms. Therefore, specific structural and functional details disclosed herein are not to be interpreted as limiting, but merely as a basis for the claims and as a representative basis for teaching one skilled in the art to variously employ the present invention in virtually any appropriately detailed structure.

Reference is now made to the drawings, wherein like characteristics and features of the present invention shown in the various DRAWINGS—FIGURES are designated by the same reference numerals.

### FIRST PREFERRED EMBODIMENT

Referring to DIAGRAM 1-A, a skin treatment mixture (8) for topical or oral application to the pierced or tattooed area of the human body, such as an area subjected to body modifications by way of piercings or tattoos in a tattoo or body-piercing studio, is disclosed.

The components are combined as follows: a quantity of non-iodized sodium chloride (1) are added to a quantity of vapor distilled water (2) until a salinity level of approximately 0.9% is reached. The non-iodized sodium chloride (1) and vapor distilled water are then mixed together and heated to 70 degrees Celsius (3) and remain at said temperature for 20 minutes to ensure proper eradication of any bacterium which may be present. The Mixture (3) is then cooled to 40 degrees Celsius (4). After mixture has cooled, Pure Aloe Barbadensis Leaf Extract is added (5) and lysozyme is added. The preferred specific proportions for the Aloe (5) and the lysozyme (6) are: aloe barbadensis leaf extract (5) is added so that a critical range between 25%-99% pure aloe product to vapor distilled water (2) is achieved. The lysozyme (6) is added so that a preferred value of 0.02% to quantity of distilled water (2) is achieved. The mixture is then mixed vigorously for 5 minutes to ensure mechanical mixture of ingredients (7). After the mixture has cooled to room temperature, we now have a finished distilled water, aloe, non-iodized sodium chloride, and lysozyme mixture (8).

### Operations—First Embodiment

In practicing the invention, the following method may be used. The preferred method steps for producing the mixture (8) include but are not limited to the following: The components are combined as follows: a quantity of non-iodized sodium chloride (1) are added to a quantity of vapor distilled water (2) until a salinity level of approximately 0.9% is reached. The non-iodized sodium chloride (1) and vapor distilled water are then mixed together and heated to 70 degrees Celsius (3) and remain at said temperature for 20 minutes to ensure proper eradication of any bacterium which may be present. The Mixture (3) is then cooled to 40 degrees Celsius (4). After mixture has cooled, Pure Aloe Barbadensis Leaf Extract is added (5) and lysozyme is added. The preferred specific proportions for the Aloe (5) and the lysozyme (6) are: aloe barbadensis leaf extract (5) is added so that a critical range between 25%-99% pure aloe product to vapor distilled water (2) is achieved. The lysozyme (6) is added so that a preferred value of 0.02% to quantity of distilled water (2) is achieved. The mixture is then mixed vigorously for 5 minutes to ensure mechanical mixture of ingredients (7). After the mixture has cooled to room temperature, we now have a finished distilled water, aloe, non-iodized sodium chloride, and lysozyme mixture (8). The mixture (8) is then either topically or orally applied depending on the treatment required.

### The Components

The non-iodized sodium chloride (1) is commonly referred to as non-iodized table salt. Non-Iodized salt is ordinary table salt that does not have iodine added to it. Many salt companies add iodine to protect against thyroid disease. But for some, iodized salt is not wanted because of the bitter taste that it can have in some recipes. Non-iodized salt is used over iodized salts for canning pickles. It is also used for fish aquariums, as iodized salt will somehow harm the fish. For smoking meat and tanning cow's hide, non-iodized salt is also used. Rock salt is also non-iodized salt and it is used to make ice cream. Non-Iodized salt is often used in cosmetics.

Vapor distilled water (2) is created when tap water is then converted to steam by a heating element. The steam is then compressed, which heats it up further to a “superheated” status. The superheated steam then travels back down into the chamber where it was originally boiled, and condenses on the now-cooled surface. Vapor distilled water is most commonly used in two settings: scientific and culinary. In the scientific realm, vapor distilled water is often made in the lab and used for experiments where water composition was be made constant. Similarly, vapor distilled water is used in drink and food recipes where slight imperfections in the water's composition would alter the flavor of the recipe.

Pure aloe barbadensis leaf extract (5) is carefully processed from the inner fillet of the aloe leaf. Care is taken to minimize disruption of the Aloin layer. Further processed to remove pulp and fiber, the aloe gel is pasteurized and concentrated utilizing low temperature evaporation. The gel is then freeze dried to produce a product without additional additives or preservatives. Freeze drying prevents the breakdown of the great majority of the long chain polysaccharides found in the fresh leaf, making this an exceptional product for cosmetic and nutraceutical applications.

The lysozyme (6), also known as muramidase or N-acetylmuramide glycanhydrolase, are glycoside hydrolases, enzymes (EC 3.2.1.17) that damage bacterial cell walls by catalyzing hydrolysis of 1,4-beta-linkages between N-acetylmuramic acid and N-acetyl-D-glucosamine residues in a peptidoglycan and between N-acetyl-D-glucosamine residues in chitodextrins. Lysozyme is abundant in a number of secretions, such as tears, saliva, human milk, and mucus. It is also present in cytoplasmic granules of the polymorphonuclear neutrophils (PMN). Large amounts of lysozyme can be found in egg white. C-type lysozymes are closely related to alpha-lactalbumin in sequence and structure, making them part of the same family.

The final mixture (8), produced as described with reference to DIAGRAM 1-A, also may be used as a throat spray. The mixture (8) can also be used to safely cleanse and heal a number of ailments including, but not limited to, tonsillitis, abscessed teeth, infected gums, and canker sores.

Although Aloe and lysozyme are abundantly available on earth, this invention represents the first attempt ever to combine those elements with non-iodized sodium chloride and vapor distilled water for the commercial purposes described herein.

While the invention has been described, disclosed, illustrated, and shown in various terms or certain embodiments or modifications which it has assumed in practice, the scope of the invention is not intended to be, nor should it be deemed to be, limited thereby and such other modifications or embodiments as may be suggested by the teachings herein are particularly reserved especially as they fall within the breadth and scope of the claims here given.

I claim as my invention:

I. A skin treatment mixture comprising: non-iodized sodium chloride, vapor distilled water, pure aloe leaf extract, and lysozyme.

II. A method of producing a skin treatment mixture of claim 1, comprising the steps of:
A. Providing a quantity of vapor distilled water.
B. Adding a quantity of non-iodized sodium chloride to the quantity of vapor distilled water.
C. Heating the vapor distilled water and non-iodized sodium chloride to 70 degrees celcius for 20 minutes.
D. Cooling the sodium chloride and distilled water to 40 degrees celcius.
E. Adding a quantity of aloe barbadensis and a quantity of lysozyme.
F. Stirring the mixture while cooling

III. A method of treating the tattooed or pierced area of a persons skin comprising the steps of: applying either topically or orally, a mixture comprising distilled water, non-iodized sodium chloride, aloe, and lysozyme.
